(19) United States
(12) Patent Application Publication
(10) Pub. No.: US 2003/0211575 A1
(43) Pub. Date: Nov. 13, 2003

(54) Constituitively open voltage-gated kand methods for discovering modulators thereof

(76) Inventors: Kenton J. Swartz, Rockville, MD (US); David H. Hackos, San Francisco, CA (US)

Correspondence Address: KLARQUIST SPARKMAN, LLP, 121 SW SALMON STREET, SUITE 1600, PORTLAND, OR, 97204, US

(21) Appl. No.: 10/203,578

(22) PCT Filed: Feb. 7, 2001
(86) PCT No.: PCT/US01/03963

(51) Int. Cl. A01K 67/00; C07H 21/04; C12N 1/18; C12P 21/02; C12N 5/06; C07K 14/47

(52) U.S. Cl. 435/69.1; 435/320.1; 435/325; 530/350; 536/23.5; 800/8; 435/254.21

(57) ABSTRACT

The present invention provides voltage-gated K⁺ channels with amino acid substitutions that produce a constitutively open phenotype when expressed in the cell. The invention includes mutated purified proteins with constitutively open voltage-gated K⁺ channel activity, for example those derived from Shaker, Shab, Shal, and Shaw family of voltage-gated K⁺ channels. The invention also includes nucleic acid encoding constitutively open voltage-gated K⁺ channels, as well as cells and transgenic animals expressing constitutively open voltage-gated K⁺ channels. Methods are provided for screening substances for ability to modulate voltage-gated K⁺ channels, by exploiting the ability of constitutively active voltage-gated K⁺ channels to affect growth or other phenotypic or genotypic characteristics in eukaryotic cells.

### CROSS REFERENCE TO RELATED APPLICATIONS

This is a §371 U.S. National Stage of International Application No. PCT/US01/03963, filed Feb. 7, 2001, which was published in English under PCT Article 21(2), and claims the benefit of U.S. Provisional Application No. 60/181,692, filed Feb. 10, 2000. Both applications are incorporated herein in their entirety.

### TECHNICAL FIELD

This invention relates to cellular physiology and pharmacology, and specifically, electrophysiology and recombinant DNA methods useful to characterize cell function and physiology. Particularly, the invention relates to the function of voltage-gated K⁺ channels, and methods for discovering inhibitors and activators of these channels.

### BACKGROUND

The surface membranes of eukaryotic cells perform essential functions, both for the life of the cell and the organism from which they were derived. Ion channels are particularly important cell surface components, since they are intimately involved in cell growth, signaling, muscle contraction, and many other processes. Numerous types of ion channels have been described, including channels for potassium, calcium, sodium, and chloride. All classes of ion channels have associated disorders that result in serious human and animal disease. See Katz, N. Engl. J. Med. 328: 1244-1251 (1993).

K⁺ channels participate in a broad spectrum of processes in excitable and non-excitable eukaryotic cells. These physiologic processes include regulation of heartbeat and vascular tone, neuronal excitability, and hormone secretion. K⁺ channel disorders may cause or exacerbate serious diseases such as epilepsy, diabetes, and sudden cardiac death. Therapy directed at K⁺ channels holds great promise in these and many other life-threatening conditions. See Nielsen-Kudsk et al., Heart 76: 109-116 (1996).

Several classes of K⁺ channels have been identified, including voltage-gated, ATP-sensitive, muscarinic-activated, Ca2⁺ activated, inward-rectifier, and outward-rectifier types. See Yost, Anesthesiology 90: 1186-1203 (1999) (hereinafter Yost). The classification is based on pharmacologic and electrophysiologic properties. Although they share many structural motifs, the classes are readily distinguishable at the ultrastructural and molecular level.

The polypeptide subunits of voltage-gated K⁺ channels are structurally characterized by a cytoplasmic amino terminal domain, six transmembrane domains designated S1-S6, and a cytoplasmic carboxyl terminal domain. See Li-Smerin et al., J. Gen. Physiol. 115: 33-50 (2000) (hereinafter Li-Smerin et al.); Chandy et al., U.S. Pat. No. 5,559,009. The ion pore is formed by S5, S6 and a pore helix in the S5/S6 linker region. Four such subunits associate noncovalently to form the channel. Transport of potassium through the channel is termed potassium conductance.

Information is available about voltage-dependent potassium conductance in excitable cells, for example in Yost. Excitable cells have a resting transmembrane electrical potential (typically about −70 millivolts), which is a function of the differential distribution of the most abundant common ions (sodium, potassium, and chloride). Potassium is considered an intracellular cation, having a 20-to-35 higher concentration inside the cell than outside. Sodium and calcium are considered extracellular cations, with much higher concentrations outside the cells.

The net movement of charged ions across a semipermeable membrane is primarily determined by the concentration gradient and electrical gradient. If the membrane potential were held artificially at zero millivolts (e.g., during a voltage-clamp experiment), there would be no electrical force and the in-to-out K⁺ concentration gradient would cause a net outward K⁺ flow. If the membrane potential were held at about −90 millivolts, the K⁺ concentration gradient and the inward electrical force would cancel each other out, and there would be no net potassium movement. This voltage, at which there is no net potassium movement, is called the potassium equilibrium potential. If the membrane potential were held artificially at −120 millivolts, the net movement of potassium would be from out-to-in.

Under various conditions of excitability, a slow inward leak of sodium and calcium causes an excitable cell's electrical potential to become less negative. At a threshold potential, typically about −40 millivolts, sodium channels rapidly open and close, allowing a transient influx of sodium. Calcium channels are also triggered, with slower on-off kinetics. As a result, the cell rapidly depolarizes, i.e., the transmembrane potential becomes transiently positive, and the excitable cell is activated. Different effects may be produced, depending on the tissue type. For example, in the heart cardiac muscle contraction occurs; in the brain neurons fire; and in endocrine tissues hormones are secreted.

Voltage-gated K⁺ channels reverse this process by repolarizing and deactivating the cell. Like sodium channels, voltage-gated K⁺ channels open transiently when the cell depolarizes to a threshold voltage. Since potassium's concentration gradient is in-to-out, the potassium flows out and opposes depolarization induced by sodium influx. The net effect is transient depolarization, followed by repolarization. Once fully repolarized, the excitable cell is ready to depolarize again.

Available techniques for the study of voltage-gated K⁺ channel include voltage clamping. In this technique, currents generated from a limited population of channels are evaluated as a function of time and voltage under varying ionic conditions. Voltage clamping may be done on a small “patch” of membrane excised with a micropipette, as in Baldwin et al, Neuron 7: 471-483 (1991), and U.S. Pat. No. 5,670,335. The technique may be adapted to study drug effects on voltage-gated K⁺ channel function, by comparing potassium conductance, on-off kinetics, or any other channel function in the presence or absence of drug. U.S. Pat. No. 5,827,655 and U.S. Pat. No. 6,013,470 provide examples of these techniques. Voltage clamping is useful for drug discovery, but is limited by inherently low throughput.

Another technique for the evaluation of K⁺ channels involves the use of the genetically engineered yeast strain SGY1528 (ATCC No. 74144) that is deleted for both of its endogenous potassium transporters, as shown in U.S. Pat. No. 5,795,770. These cells grow poorly in low-potassium media, but their growth may be rescued by transfection of a heterologous plant K⁺ channel. Compounds that inhibit the heterologous plant K⁺ channel will cause the transfected SGY1528 cells to revert to impaired growth in low-potassium media. Transfection of a mammalian inward-rectifying K⁺ channel also allows SGY1528 to grow in low-potassium media, as in U.S. Pat. No. 5,620,892 and Tang et al, Mol. Biol. Cell 6:1231-1240 (1995). However, the strategy of rescuing low-potassium SGY1528 growth is not well-suited to voltage-gated K+ channels, because these channels are closed at resting membrane potentials seen in yeast.

A better understanding of voltage-gated K⁺ channels is needed to design improved methods of screening for substances that modulate voltage-gated K⁺ channels. Such modulating substances would have great therapeutic potential in numerous debilitating and life-threatening conditions, for example in the treatment of cardiac rhythm disturbances, diabetes, hypertension, asthma, and seizure disorders. See Katz, N. Engl. J. Med. 328:1244-1251 (1993) Neilsen-Kudsk, Heart 76: 109-116 (1996); Rasmusson et al. Circulation Research 82: 739-750 (1998). In addition, K⁺ channel modulators may form a new class of general anesthetic agents, as discussed in Yost.

### SUMMARY OF THE DISCLOSURE

The foregoing problems are addressed by the present invention, wherein mutations that produce a constitutively open voltage-gated K⁺ channel have been found. These and other mutations can be used in methods to screen for and discover useful voltage-gated K⁺ channel modulators.

The molecular basis of this precise regulation of cation movement in excitable cells is beginning to be understood. The S4 segment transmembrane domain of voltage-gated K⁺ channels contains a motif rich in positively charged amino acid residues, which is believed to be an important voltage sensor. The S2 and S3 segments may also be involved in voltage sensing. Moreover, S1 is present in all voltage-gated K₊ channels and likely participates in voltage sensing. Thus, it is reasonable to regard the entire S1-S4 segment as the voltage-sensing domain.

When the cell reaches threshold voltage, the S1-S4 voltage sensor rearranges, inducing a conformational change that opens the channel pore formed by parts of the S5 and S6 domains from the four subunits. The channel is then inactivated by two distinct molecular mechanisms. Rapid inactivation occurs through interaction of the globular amino terminus with the ion-conducting pore. Slow inactivation, which is believed to occur by rearrangement of the channel's four protein subunits, leads to narrowing of the mouth of the pore.

The present invention takes advantage of the discovery that mutations in voltage-gated K⁺ channels can produce a constitutively open phenotype, in which the mutated voltage-gated K⁺ channel conducts potassium ions under conditions in which unmutated voltage-gated K⁺ channels do not. The invention includes mutated purified proteins with constitutively open voltage-gated K⁺ channel activity, for example mutated proteins derived from the Shaker, Shab, Shal, and Shaw family of voltage-gated K⁺ channels, or from the eag, elk, or erg subfamilies of voltage-gated K⁺ channels. The mutated purified proteins may originate as Drosophila voltage-gated K⁺ channels, or alternatively, as voltage-gated K⁺ channels from any mammal, such as mouse, rat, or human. In some embodiments, the mutations may be in the S5 or S6 transmembrane segments, or the S5/S6 linker which includes the pore helix. In other embodiments, the mutations may be in the S1, S2, S3 and S4 transmembrane segments, which is believed to be the voltage-sensing domain of voltage-gated K⁺ channels.

In particular embodiments, the mutations may be in an amino acid residue of the PVP motif of the voltage-gated K⁺ channel, for example mutations that change the first or second Pro of the PVP motif, for example the second Pro, to Asp Glu, Lys, His, Asn, or Gln. In other embodiments, the mutations change the second Pro of the PVP motif to Arg, Tyr, Phe, Trp, Met, Gly, Val, Ile, Cys, Ser, or Ala. In some embodiments, Pro 475 of a Shaker family voltage-gated K⁺ channel is mutated, whereas in others, Pro 406 of a Shab family channel, Pro 403 or 404 of a Shal family channel, or Pro 410 of a Shaw family channel is mutated. In other embodiments, the mutations may substitute an amino acid for the first Pro of the PVP motif, or may substitute an amino acid residue other than valine or cystine for the central Val of the PVP motif. In some embodiments, the mutations produce a constitutively open phenotype in the substantial absence of cadmium.

The present invention also includes cells expressing constitutively open voltage-gated K⁺ channels, for example Xenopus oocytes expressing constitutively open voltage-gated K⁺ channels. In some embodiments, the cell's growth in low-potassium media may be enhanced by expression of the constitutively open voltage-gated K⁺ channel. The cell may be a yeast cell, for example Saccharomyces cerevisiae, and the Saccharomyces cerevisiae cell may have impaired function of the trk1-trk2 potassium transporters.

The present invention also includes methods for testing a substance to determine whether it modulates voltage-gated K⁺ channel function. This method includes providing a cell expressing a constitutively open voltage-gated K⁺ channel, treating the cell with a substance, and detecting any phenotypic or genotypic characteristic of the cell. The detected characteristic may be voltage-gated K⁺ channel function, for example potassium conductance, steady-state current, tail current, voltage-gating, channel activation, and channel inactivation. In some embodiments, the detected characteristic is cell growth, for example growth of a yeast cell, such as a Saccharomyces cerevisiae cell having impaired function of the trk1-trk2 potassium transporters.

The present invention also includes compounds identified as having ability to modulate voltage-gated K⁺ channel function using the methods of this invention.

The present invention also includes recombinant nucleic acid molecules having a promoter sequence operably linked to a voltage-gated K⁺ channel that has been mutated to produce a constitutively open voltage-gated K⁺ channel upon expression in a cell. In some embodiments, the recombinant nucleic acid molecules may be derived from the Shaker, Shab, Shal, or Shaw family of voltage-gated K⁺ channels. In other embodiments, the recombinant nucleic acid molecule may be derived from a nucleic acid encoding a voltage-gated K⁺ channel that includes a PVP motif. In yet other embodiments, the recombinant nucleic acid molecule may contain a mutation at the second Pro of the PVP motif, for example a mutation encoding Asp, Glu, Lys, His, Asn, Gln, Arg, Tyr, Phe, Trp, Met, Gly, Val, Ile, Cys, Ser, or Ala.

The invention also includes cells transformed with mutated nucleic acids expressing constitutively open voltage-gated K⁺ channels, as well as transgenic animals having recombinant nucleic acid molecules encoding constitutively open voltage-gated K⁺ channels.

The foregoing and other objects, features, and advantages of the invention will become more apparent from the following detailed description of several embodiments, which proceeds with reference to the accompanying figures.

### BRIEF DESCRIPTION OF THE FIGURES

FIG. 1 shows structural aspects of voltage-gated K⁺ channels, with particular emphasis on the location and function of the channel pore. FIG. 1A illustrates the structure of a single voltage-gated K⁺ channel subunit. FIG. 1B illustrates a possible arrangement of the S1-S6 helices when four of the channel subunits are arranged as a tetramer to form a functional voltage-gated K⁺ channel. This is illustrated as a cross-section through domains S1-S6, shown from an extracellular perspective. FIG. 1C illustrates pore domain structure, and aligns primary sequences of pore domains from the bacterial KcsA K⁺ channel (SEQ ID NO:1) with four classes of voltage-gated K⁺ channels. Two representative members of each of the four classes include Shaker (SEQ ID NO:2) and Kv1.1 (SEQ ID NO:3), Shab (SEQ ID NO:4) and Kv2.1 (SEQ ID NO:5), Shaw (SEQ ID NO:6) and Kv3.1 (SEQ ID NO:7), and Shal (SEQ ID NO:8) and Kv4.2 (SEQ ID NO:9).

FIG. 2 shows current records from a Xenopus oocyte expressing wild-type Shaker channels studied with two-electrode voltage clamp. FIG. 2A (top) shows voltage as a function of time. FIG. 2A (bottom) shows current recorded as a function of time, at voltages shown in FIG. 2A, top. FIG. 2B shows normalized tail currents plotted as a function of voltage. FIG. 2C shows steady-state current plotted as a function of test voltage. The voltages used are those presented in FIG. 2A. Steady-state currents are also those shown in FIG. 2A, measured during the plateau of current late in the test depolarization, just prior to repolarization to −60 mV.

FIG. 3 shows current records from a Xenopus oocyte expressing mutant Shaker channels harboring a P475D mutation, studied with a two-electrode voltage clamp. The applied voltage as a function of time is shown in FIG. 3A, top. FIG. 3A (bottom) shows current recorded as a function of time, at voltages shown in FIG. 3A, top. FIG. 3B presents steady-state current as a function of the applied depolarizing/hyperpolarizing voltage.

FIG. 4 presents voltage-current data for the Shaker P475E mutant.

FIG. 5 presents voltage-current data for the Shaker P475K mutant.

FIG. 6 presents voltage-current data for the Shaker P475H mutant.

FIG. 7A presents voltage-current data for the Shaker P475N mutant. FIG. 7B presents tail currents observed with P475N, recorded 2 msec after return from test depolarizing voltage to −150 mV

FIGS. 8A-B present voltage-current and tail current data for the Shaker P475Q mutant.

FIG. 9A presents voltage-dependent gating in the wild type Kv2.1 K⁺ channel. FIG. 9B presents voltage-current data from an oocyte expressing a Kv2.1 K⁺ mutant, P406D. FIG. 9B, top, shows that membrane voltage was held at −110 mV, depolarized to different voltages, and returned to −110 mV to elicit a tail current. FIG. 9C compares tail current activation curves for P406D and wild type Kv2.1 K channels. Normalized tail currents are plotted against test voltage for the two Xenopus oocytes shown in FIGS. 9A and 9B.

### Sequence Listing

The amino acid sequences listed in the accompanying sequence listing are shown using the standard three letter code for amino acids.

SEQ ID NO:1 shows the amino acid sequence of the pore domains of the bacterial KcsA K⁺ channel.

SEQ ID NO:2 shows the amino acid sequence of the pore domains of the drosophila Shaker K⁺ channel.

SEQ ID NO:3 shows the amino acid sequence of the pore domains of the rat Kv1.1 K⁺ channel.

SEQ ID NO:4 shows the amino acid sequence of the pore domains of the drosophila Shab K⁺ channel.

SEQ ID NO:5 shows the amino acid sequence of the pore domains of the rat Kv2.1 K⁺ channel.

SEQ ID NO:6 shows the amino acid sequence of the pore domains of the drosophila Shaw K⁺ channel.

SEQ ID NO:7 shows the amino acid sequence of the pore domains of the rat Kv3.1 K⁺ channel.

SEQ ID NO:8 shows the amino acid sequence of the pore domains of the drosophila Shal K⁺ channel.

SEQ ID NO:9 shows the amino acid sequence of the pore domains of the rat Kv4.2 K⁺ channel.

### DETAILED DESCRIPTION OF SEVERAL ILLUSTRATIVE EMBODIMENTS

### Definitions

Animal: Living multi-cellular vertebrate organisms, a category that includes, for example, mammals and birds. The term mammal includes both human and non-human mammals. Similarly, the term “subject” includes both human and veterinary subjects.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Constitutively open voltage-gated K⁺ channel: a voltage-gated K⁺ channel harboring a mutation that at one or more membrane voltages results in increased potassium conductance relative to a unmutated voltage-gated K⁺ channel. In many instances, a constitutively open voltage-gated K⁺ channel has detectable potassium conductance at voltages below threshold voltage for the unmutated voltage-gated K⁺ channel. A typical threshold voltage for an unmutated voltage-gated K⁺ channel would be about −40 millivolts. At voltages more negative than the threshold voltage, an unmutated voltage-gated K⁺ channel allows substantially no potassium conductance, whereas a constitutively open voltage-gated K⁺ channel allows a greater detectable amount of potassium conductance.

Depolarize: induce the membrane voltage to become more positive or less negative.

Eag, elk, and erg subfamilies of voltage-gated K⁺ channels: Subfamilies of genes that encode potassium ion channel proteins have been identified in and isolated from, various organisms including C. elegans, Drosophila, mouse, rat, and humans. The members of the gene family are classified into subfamilies (eag, elk, and erg) on the basis of sequence similarity. The Eag family is named for the original isolate from Drosophila (ether-a go-go or eag). The Elk family includes genes that encode eag-like potassium ion channels. The Erg family includes eag-related genes.

Typically there is about 40-50% amino acid identity among proteins in the different subfamilies. Within a subfamily, the amino acid identity is about 60-70% even for proteins from different species. The human Erg ion-channel gene (Herg) corresponds to the LQT-2 genetic locus and maps to chromosome 7 q-35-36. Mutations in Herg can cause long-QT (LQT) syndrome, a relatively rare disorder that causes syncope and sudden death due to ventricular arrhythmia. The characteristic increased electrocardiographic Q-T interval evidences delayed repolarization of the cardiac action potential. At a molecular level, the delayed repolarization is linked with abnormal ion channel behavior. The Herg gene, in particular, is shown to be altered or defective in both acquired and inherited forms of LQT syndrome.

Herg ion channels are inwardly rectifying potassium channels. Herg channels have properties consistent with the gating properties of eag, and other, outwardly-rectifying, S4-containing potassium channels, but with the addition of an inactivation mechanism that attenuates potassium efflux during depolarization. It is thought that these properties of Herg channel function are critical to maintaining normal cardiac rhythmicity. The molecular mechanism by which Herg ion channels protect the heart against inappropriate rhythmicity is elucidated in Smith, P. L., et al., “The Inward Rectification Mechanism of the HERG Cardiac Potassium Channel,” 379 Nature 33 (1996) and in Miller, C. “The Inconstancy of the Human Heart,” 379 Nature 767 (1996).

The Herg (“human ether-a-go-go”) gene that encodes the human Herg potassium ion channel subunits was described by Warmke and Ganetzky, 91 PNAS USA 3438-3442 (1994), incorporated herein by reference. Human eag sequences are found at Genbank Accession Number NM002238 and AF078741. The Herg DNA sequence is found at Genbank Accession Number U04270. A Drosophila erg gene was described by Titus et al., 17 J. Neuroscience 875-881 (1997) and is reported at Genbank Accession Number U42204. A C. elegans erg gene is found at Accession Numbers U02425 and U02453 of the Genbank database. Elk subfamily sequences are found at Genbank Accession Numbers AF073892 and AF061957 (rat).

Homologs: two nucleotide sequences that share a common ancestral sequence and diverged when a species carrying that ancestral sequence split into two species.

Hyperpolarize: induce the membrane voltage to become more negative.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Mutant: A mutation may include nucleotide sequence changes (substitutions), additions (insertions) or deletions, including deletion of large portions of the sequence. The effect of such mutations can be determined by the assay of the present invention in which K⁺ conductance into a cell is determined.

Nucleic acid: A deoxyribonucleotide or ribonucleotide polymer in either single or double stranded form, and unless otherwise limited, encompasses known analogues of natural nucleotides that hybridize to nucleic acids in a manner similar to naturally occurring nucleotides.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Polypeptide: any chain of amino acids, regardless of length or post-translational modification (e.g., glycosylation or phosphorylation).

Pore domain: amino acid residues of a voltage-gated K+ channel that form the channel's pore through which cations are conducted. In Shaker, Shab, Shal, and Shaw family channels, the pore domain is made up of the S5 and S6 transmembrane segments, and a pore helix in the S5/S6 linker region.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

PVP motif: a conserved amino acid sequence of Pro-Val-Pro or Pro-Ile-Pro found in the sixth transmembrane domain (S6) of voltage-gated K+ channels. The first Pro of the PVP motif is defined as the Pro closest to the amino terminus of the polypeptide chain, and the second Pro of the PVP motif is defined as the Pro closest to the carboxy terminus of the polypeptide chain.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded for by a recombinant nucleic acid molecule.

Repolarize: return to more negative membrane voltages after the membrane voltage has become less negative or more positive. Eukaryotic cells typically repolarize by opening of voltage-gated K+ channels, allowing an outward potassium current that makes the interior of the cell more negative. Other channels also contribute to repolarization, such as other types of K+ channels.

Reversal potential (or Reversal voltage, Vᵣₑᵥ): the membrane voltage at which net current is 0.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of the MB1 protein, and the corresponding cDNA or gene sequence, will possess a relatively high degree of sequence identity when aligned using standard methods. This homology will be more significant when the orthologous proteins or genes or cDNAs are derived from species that are more closely related (e.g., human and chimpanzee sequences), compared to species more distantly related (e.g., human and C. elegans sequences).

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene, 73: 237-244, 1988; Higgins & Sharp CABIOS 5: 151-153, 1989; Corpet et al. Nuc. Acids Res. 16, 10881-90, 1988; Huang et al. Computer Appls. in the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website, together with a description of how to determine sequence identity using this program.

Homologs of the disclosed human channel proteins typically possess at least 60% sequence identity counted over full-length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, at least 75%, at least 80%, at least 90%, at least 95%, at least 98%, or at least 99% sequence identity. When less than the entire sequence is being compared for sequence identity, homologs will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI website.

One of ordinary skill in the art will appreciate that these sequence identity ranges are provided for guidance only; it is entirely possible that strongly significant homologs could be obtained that fall outside of the ranges provided. The present invention provides not only the peptide homologs that are described above, but also nucleic acid molecules that encode such homologs.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tm) for the specific sequence at a defined ionic strength and pH. The Tm is the temperature (under defined ionic strength and pH) at which 50% of the target sequence remains hybridized to a perfectly matched probe or complementary strand. Conditions for nucleic acid hybridization and calculation of stringencies can be found in Sambrook et al. ((1989) In Molecular Cloning: A Laboratory Manual, CSHL, New York) and Tijssen ((1993) Laboratory Techniques in Biochemistry and Molecular Biology—Hybridization with Nucleic Acid Probes Part I, Chapter 2, Elsevier, N.Y.). Nucleic acid molecules that hybridize under stringent conditions to a human encoding sequence will typically hybridize to a probe based on either an entire human encoding sequence or selected portions of the encoding sequence under wash conditions of 2×SSC at 50° C.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid molecules that all encode substantially the same protein.

Substantially similar nucleotide sequences, when optimally aligned (with appropriate nucleotide insertions or deletions) with a comparison nucleic acid (or its complementary strand), have a nucleotide sequence identity in at least about 50%, 60%, 70%, 80%, 90%, 95% or 98% of the nucleotide bases.

Shaker, Shab, Shaw, and Shal families of voltage-gated K+ channels: Four related genes, Shaker (GenBank accession number X78908), Shab (GenBank AF084525), Shaw (GenBank M32661), and Shal (GenBank M32660), encode voltage-gated K+ channels in Drosophila. Shaker and Shal encode voltage-gated potassium channels with rapid current activation and inactivating properties. Shab and Shaw encode delayed rectifier channels, with slow inactivating (Shab) and non-inactivating (Shaw) properties.

Multigene subfamilies corresponding to each of these Drosophila genes have been identified, rodents, primates, other mammals, and humans (e.g., GenBank accession numbers NM 10596; NM 004974; NM 000217; NM 00233-00235; U89873; NM 008423; NM 004980; NM 004979; NM 008436; NM 004975; NM 004770). The multigene subfamilies suggest that the four genes are older than the common ancestor of present-day insects and mammals and that the expansion of each into a family occurred before the divergence of rodents and primates. In Drosophila, subfamily heterogeneity is derived by alternative splicing, while in mammals diversity among channel subtypes is generated primarily by distinct genes. Further diversity of mammalian potassium channels is demonstrated by the identification of some that do not belong to any of the four main subfamilies.

The diversity of voltage-gated K+ channels leads to variation in the location of particular amino acid residues, even within subfamilies. For example, in the Shab subfamily, the PVP motif of the S6 transmembrane domain is located at amino acid residue number 404-406 in Kv2.1 from rat (Frech et al., Nature. 340:642-5, 1989), but is located at 654-656 in Drosophila Shab (Butler et al., Science 243:943-946, 1989). In other cases, the differences in numbering are less striking. For example, in Drosophila Shal, the PVP motif is located at amino acid residues 402-404, whereas the PVP motif is located at 401-403 in rat Kv4.2. Baldwin et al., Neuron 7:471, 1991. Such variations are well known to those skilled in the art. Members of subfamilies are readily identified as such by homology of nucleic acid and primary amino acid sequence.

Steady-state current: the current observed through a voltage-gated K+ channel when voltage is held constant.

Tail current: a current relaxation caused by the closing of voltage-gated K+ channels when voltage is stepped from a voltage where the open probability is high to a voltage where the open probability is low. The peak of the tail current will have an amplitude proportional to the fraction of channels open at the preceding voltage. For example, at a voltage of 0 mV, the open probability of a Shaker voltage-gated K⁺ channel is high. If the voltage is abruptly shifted to −50 millivolts, the open probability becomes low, and a tail current is observed during the time required for the channel to transition from open to closed. Tail current measurement allows kinetic evaluation of voltage-dependent gating.

Transduced and Transformed: A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transfer vector: A vector which shuttles a transgene.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Viral Vectors: a vector that is derived from a virus, such as adenoviruses, adeno-associated viruses, vaccinia, herpes simplex virus, alpha virus, lentivirus, and retrovirus vectors.

Voltage sensing domain: the S1, S2, S3, and S4 transmembrane domains of voltage-gated K⁺ channels, involved in sensing membrane voltage changes and altering the channel's cation conducting characteristics in response to these changes.

Voltage-gated K⁺ channel: Voltage-gated potassium (K⁺) channels are assembled by four identical or homologous alpha-subunits to form a tetrameric complex with a central conduction pore for potassium ions. Most of the cloned genes for the alpha-subunits are classified into four subfamilies: Kv1, also known as Shaker; Kv2, also know as Shab; Kv3, also known as Shaw; and Kv4, also known as Shal. Typically, the subunits have a cytoplasmic amino terminal domain; four transmembrane domains S1, S2, S3, and S4 believed to function as voltage sensors; two transmembrane domains S5 and S6, believed to combine with the S5/S6 linker region (also known as the pore helix) to form the pore domain through which cations are conducted; and a carboxyl terminal cytoplasmic domain

Voltage-gated K⁺ channel activator: a substance that increases potassium conductance at any membrane voltage, increases steady-state and/or tail currents at any membrane voltage, makes channel inactivation less likely at any membrane voltage, and/or makes channel activation more likely at any membrane voltage.

Voltage-gated K⁺ channel inhibitor: a substance that reduces potassium conductance at any membrane voltage, reduces steady-state and/or tail currents at any membrane voltage, makes channel activation less likely at any membrane voltage, and/or makes channel inactivation more likely at any membrane voltage.

Voltage-gated K⁺ channel modulator: either a voltage-gated K⁺ channel activator or a voltage-gated K⁺ channel inhibitor. Note that it is conceivable that a substance could be both a voltage-gated K⁺ channel activator and inhibitor, e.g., enhances potassium conductance at negative membrane voltages, but makes channel activation less likely at voltages around threshold potential.

Unless otherwise defined, all technical and scientific terms used herein have the same meaning as commonly understood by one of ordinary skill in the art to which this invention belongs. Also, the singular (such as “a” or “the”) includes the plural, unless context clearly indicates otherwise. Although methods and materials similar or equivalent to those described herein can be used in the practice or testing of the present invention, suitable methods and materials are described below. However, the materials, methods, and examples are illustrative only and are not intended to be limiting.

### EXAMPLE 1

### Sequence Alignment of the Pore Domains of K+ Channels

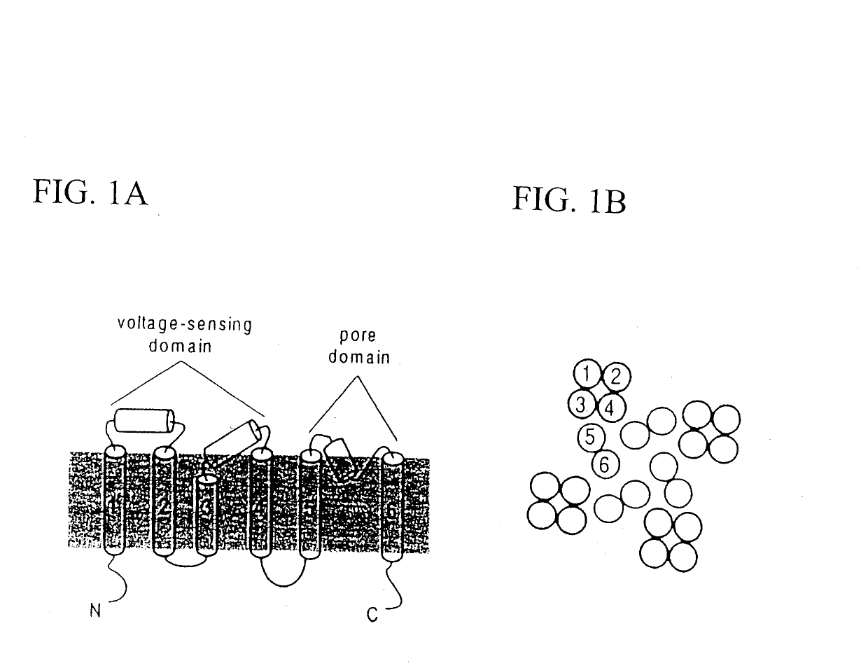

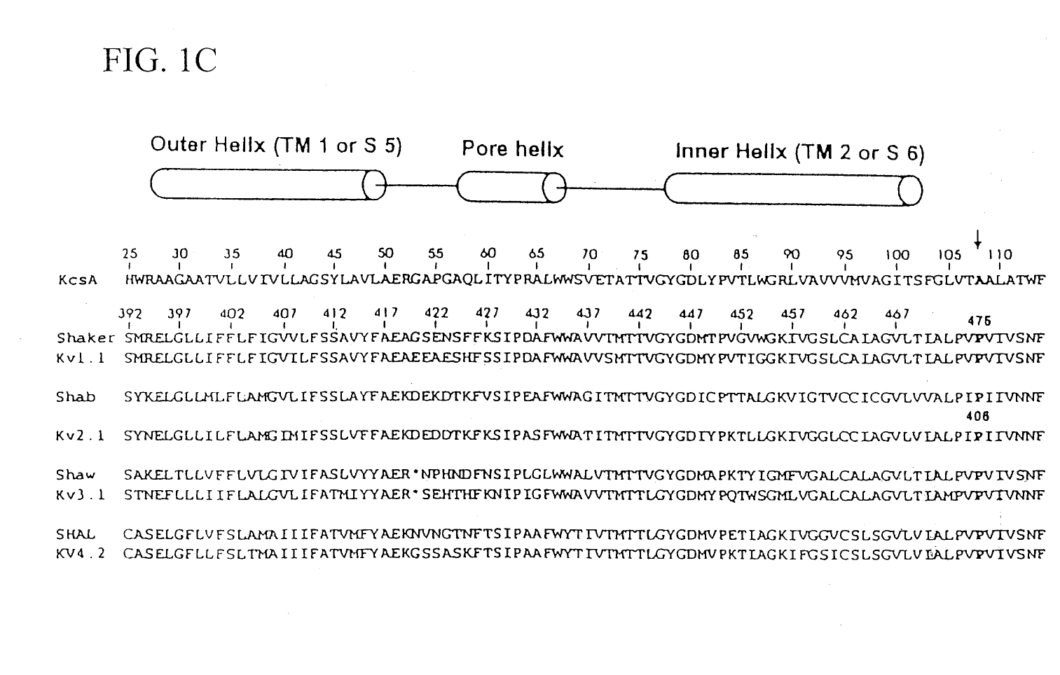

FIG. 1 shows structural aspects of voltage-gated K+ channels, with particular emphasis on the location and function of the channel pore.

FIG. 1A illustrates the structure of a single voltage-gated K+ channel subunit. There are six membrane spanning α-helical segments designated S1, S2, S3, S4, S5, and S6. The structure also contains a short pore helix, between S5 and S6, and two possible α-helices in the S1-S2 and S3-S4 extracellular linkers. S1-S4 may be regarded as a voltage-sensing domain, whereas a pore domain includes S5, S6, and the pore helix. See Li-Smerin et al. J. Gen. Physiol., 115: 33-50, 2000 (hereinafter Li-Smerin et al.); and U.S. Pat. No. 5,559,009.

FIG. 1B illustrates a proposed arrangement of the S1-S6 helices when four of the channel subunits shown in FIG. 1A are arranged as a tetramer to form a functional voltage-gated K⁺ channel. The tetramer is illustrated as a cross-section through domains S1-S6, shown from an extracellular perspective.

FIG. 1C illustrates pore domain structure, and aligns primary sequences of pore domains from the bacterial KcsA K⁺ channel with four classes of voltage-gated K⁺ channels. Diagram above amino acid sequences indicates secondary structural motifs in KcsA. TM1 and TM2 in KcsA correspond to S5 and S6 in the voltage-gated K⁺ channels, respectively. Placement of helical transmembrane domains S5 and S6, and the pore helix illustrated extending there between, is derived from the known atomic structure of the KcsA K⁺ channel, and Kyte-Doolittle hydrophobicity analysis. Kyte and Doolittle, J. Mol. Biol. 157-132, 1982.

A conserved Pro, shown in bold font and indicated by an arrow, occupies position 475 in Shaker and 406 in Kv2.1 (a Shab subfamily member obtained from rat). Asterisk (*) indicates that 3 (Shaw) and 10 (Kv3.1, Shaw subfamily member obtained from rat) residues have been deleted for purposes of alignment.

### EXAMPLE 2

### Mutagenesis and Channel Expression

The Shaker H4 K⁺ channel cDNA (Kamb et al., Neuron 1: 421-430, 1988) in a pBluescript expression vector (Stratagene, La Jolla Calif.) was modified by deleting part of the N terminus (Δ6-46) to remove fast inactivation by Hoshi et al. (Science 250: 533-538, 1990). Point mutations were introduced into this modified Shaker H4 K⁺ channel using standard PCR-based mutagenesis techniques known to those with skill in the art. See, e.g., Kumar, U.S. Pat. No. 5,556,747; Shimada, Methods in Molecular Biology 57: 157-165. PCR fragments encoding mutations were ligated into the appropriately digested pBluescript vectors, and verified by automated DNA sequencing.

Point mutations were made in the Kv2.1 K⁺ channel (a Shab family member encoding a delayed rectifier-type voltage-gated K⁺ channel from rat; Frech et al., Nature 340: 642-645, 1989) as described for the Shaker K⁺ channel using a construct in a pBluescript vector that contained several previously introduced unique restriction sites (Swartz and MacKinnon, Neuron 18, 675-82, 1997). cDNAs encoding wild-type and mutant channels Shaker and Kv2.1 K⁺ channels were linearized with HindIII and NotI, respectively, and transcribed with T7 RNA polymerase to make cRNA for cellular injection.

Oocytes were removed surgically from Xenopus laevis frogs and defolliculated by incubating with agitation for 1-1.5 hrs in a solution containing (mM): NaCl (82.5), KCl (2.5), MgCl₂ (1), HEPES (5), collagenase (2 mg/ml) and adjusted to pH 7.6 with NaOH. Defolliculated oocytes were injected with cRNA and incubated at 17° C. in a solution containing (mM): NaCl (96), KCl (2), MgCl₂ (1), CaCl₂ (1.8), HEPES (5), gentamicin (50 μg/ml; Gibco BRL), pH 7.6 with NaOH.

In addition to Xenopus oocytes, numerous other suitable cell types may be used. Chinese Hamster Ovary (CHO) and human embryonic kidney (HEK 293) cells are commonly used by those skilled in the art. A broad range of other eukaryotic cells and cell lines are also appropriate for methods of studying K⁺ conductance, and screening for modulators of K⁺ channel activity. The nucleic acid may be, for example, injected mRNA, injected cRNA, or plasmid DNA encoding the constitutively active voltage-gated K⁺ channel subunits operably linked to a promoter. Numerous other approaches are possible, such as transfection with viral vectors capable of expressing the mutated channel.

### EXAMPLE 3

### Electrophysiology of Shaker H4 K⁺ Channels

Channels were studied using two-electrode voltage clamp recording between 1 and 5 days after cRNA injection using an OC-725C oocyte clamp (Warner Instruments, Hamden Conn.). Oocytes were studied in a 160 μl recording chamber that was perfused with a solution containing (mM): RbCl (50), NaCl (50), MgCl₂ (1), CaCl₂ (0.3) and HEPES (5), pH 7.6 with NaOH. Data were filtered at 2 kHz (8-pole Bessel) and digitized at 10 kHz. Microelectrode resistances were between 0.2-1.2 MΩ when filled with 3 M KCl. All experiments were performed at room temperature (˜22° C.).

Voltage-activation relations were obtained by measuring tail current amplitude for various strength depolarizations using 50 mM Rb⁺ as the charge carrier to slow deactivation. The reversal potential under these ionic conditions was approximately −25 mV. Holding voltages were chosen where no steady-state inactivation could be detected, typically −130 to −80 mV. For most channels, inward tail currents were elicited by repolarization to −50 mV and tail current amplitude measured 1-3 ms after repolarization. More negative tail voltages were used for mutants with large negative shifts in the voltage-activation relationship. For mutants with large rightward shifts in the voltage-activation relationship, outward tail currents were elicited by repolarization to voltages between 0 and +30 mV. For a few mutant channels that displayed fast deactivation kinetics, conductance-voltage (G-V) relations were also examined using steady-state outward K⁺ currents and calculating G according to G=I/V−Vᵣₑᵥ. In these instances, tail voltage-activation relations and G-V relations were comparable.

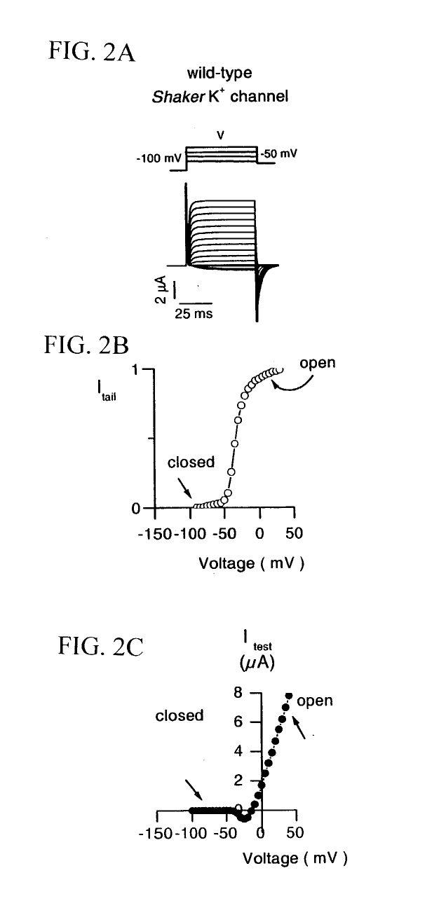

FIG. 2 shows current records from a Xenopus oocyte expressing wild-type Shaker channels studied with two-electrode voltage clamping. FIG. 2A (top) shows voltage as a function of time. Initially, the voltage is clamped at −100 millivolts. Then a pulse is applied to raise the voltage to levels near or above the cell's depolarizing threshold (in successive experiments, to about −10 millivolts, −20 millivolts, −30 millivolts, and −40 millivolts). The pulse is applied for about 50 milliseconds, and the voltage is then returned to −60 millivolts. While the voltage is being manipulated, the current is being recorded as a function of time (FIG. 2A, bottom). At −100 millivolts, there is no detectable current, indicating that the expressed Shaker K⁺ channel is completely closed. A strong depolarizing voltage pulse, e.g. to −10 millivolts, induces a positive current that rapidly rises to a plateau of about 6 μA. The current remains at 6 μA for about 50 milliseconds while the voltage is clamped at −10 millivolts. When the voltage is returned to −60 millivolts, the current becomes transiently negative (referred to as a tail current), then returns to 0 μA. The absence of a positive current under these ionic conditions indicates that the K⁺ channel is again closed.

FIG. 2B shows normalized tail currents plotted as a function of voltage. Tail currents were measured 2 msec after voltage was returned to −60 millivolts from the test depolarizing voltage. Tail currents were normalized by assigning the largest observed tail current a value of 1.0, and expressing other tail currents as a decimal fraction of the largest observed tail current.

No tail current is observed until the test depolarizing voltage exceeds −50 millivolts. As the test depolarizing voltage becomes less negative, the tail current rises steeply until the test depolarizing voltage approaches −10 millivolts. The tail current then plateaus until it reaches its maximum at the test depolarizing voltage of +40 millivolts. These data confirm that the expressed Shaker K⁺ channel remains closed at voltages more negative than −50 millivolts.

FIG. 2C shows steady-state current plotted as a function of test voltage. The voltages used are those presented in FIG. 2A. Steady-state currents are also those shown in FIG. 2A, measured during the plateau of current late in the test depolarization, just prior to repolarization to −60 mV. At voltages more negative than −5 mV, the steady-state current is zero, because channels are closed. With depolarizations less negative than −50 mV but more negative than −20 millivolts, a current activates and is inward (negative). This reflects partial channel opening, allowing inward Rb⁺ flow that modestly exceeds outward K⁺ flow. The current reverses at about −20 mV. This is the reversal potential under the test ionic conditions of 50 mM Rb⁺ as the charge carrier in the external solution, and 100 mM K⁺ as the charge carrier in the internal solution. As depolarizing voltage exceeds −20 millivolts, the net current is outward (positive), reflecting a large outward potassium flow as the channel fully opens.

### EXAMPLE 4

### Electrophysiology of Mutant Shaker Channels

This example describes electrophysiological behavior of several Shaker H4 mutants. These are mutants that produce a constitutively open phenotype, i.e., a channel that does not completely close, even at voltages far below that which would close the wild-type channel. The mutations involve Pro 475 (P475), a pore residue in the S6 transmembrane segment of the Shaker channel. Amino acid substitutions at P475 that produce a constitutively open phenotype include: Asp (P475D), Glu (P475E), Lys (P475K), His (P475H), Asn (P475N), and Gln (P475Q). However, amino acid substitutions at P475 that resulted in a channel that could close completely include: Arg (P475R), Tyr (P475Y), Phe (P475F), Trp (P475W), Thr (P475T), Met (P475M), Gly (P475G), Val (P475V), Ile (P4751), Cys (P475C), Ser (P475S), and Ala (P475A).

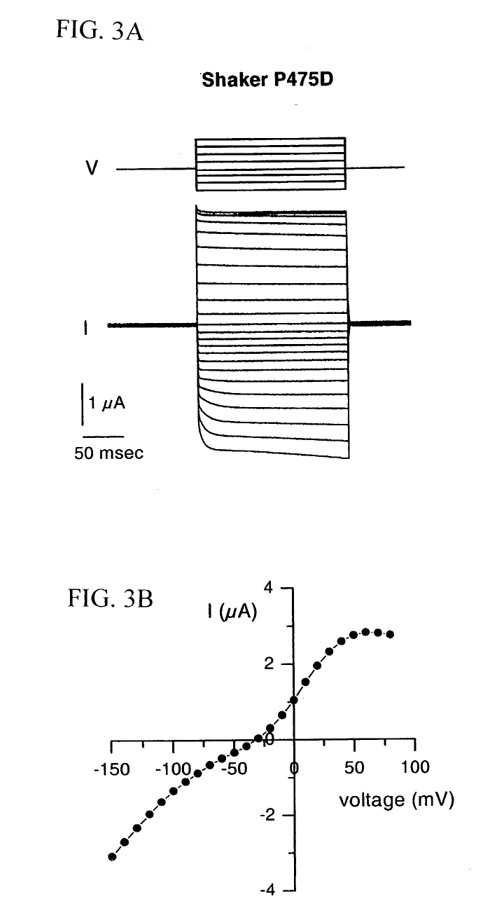

The P475 mutations were produced according to the methods described in Example 2, and electrophysiology studies were conducted according to the methods described in Example 3. FIG. 3 is analogous to FIG. 2, except that FIG. 3 shows results from P475D, whereas FIG. 2 shows results from the wild-type Shaker H4 channel. FIG. 3 documents that the P475D mutation produces a constitutively open voltage-gated K⁺ channel.

The applied voltage as a function of time is shown in FIG. 3A, top. Resting voltage is −30 millivolts; from resting voltage a depolarizing (above baseline, less negative) or a hyperpolarizing (below baseline, more negative) pulse was applied. The current traces in FIG. 3A, bottom, were induced by the depolarizing/hyperpolarizing voltages shown in FIG. 3A top.

FIG. 3A shows that large currents are observed for hyperpolarization to negative voltages that would normally close the wild type channel. In comparison, FIG. 2A shows that native Shaker channels allow no inward (negative) potassium current, even at potentials as negative as −100 millivolts. Thus, the channels illustrated in FIG. 2A are closed (if they were not closed, the strong negative potential would result in net inward K⁺ flow, under the disclosed ionic conditions). In contrast, FIG. 3A shows strong inward (negative) potassium currents at negative potentials, indicating that the channel fails to close. Indeed, depolarizations as strong as −150 millivolts do not produce channel closing, but simply result in greater inward currents. Thus, the P475D voltage-gated K⁺ channel is constitutively open.

Background and leak currents have been subtracted from the Shaker K⁺ channel current by blocking Shaker with saturating concentrations (1 μM) of agitoxin2 and then subtracting the unblocked currents (leak and background currents) from the total currents measured in the absence of agitoxin2. Thus the currents observed at voltages as negative as −150 mV are not leak current but are currents arising from the Shaker K⁺ channel. See Garcia et al., Biochemistry 33: 6834-6839 (1994).

FIG. 3B presents steady-state current as a function of the applied depolarizing/hyperpolarizing voltage. Currents were measured 10 msec after changing from the resting voltage of −30 millivolts to the test voltage. FIG. 3B is analogous to FIG. 2C. In FIG. 2C negative voltages produced 0 current, whereas in FIGS. 3A and 3B negative voltages produced large inward (negative) currents. This is further evidence that the P475D voltage-gated K⁺ channel is constitutively open.

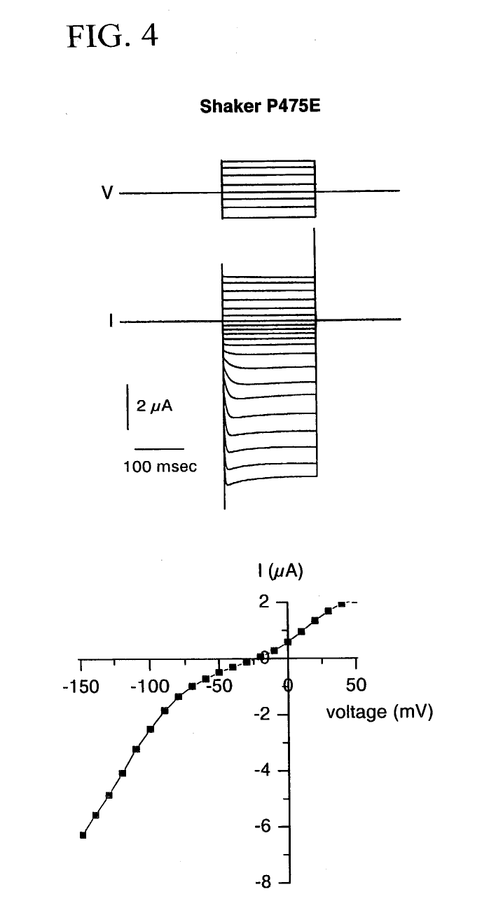

FIG. 4 presents voltage-current data for the Shaker P475E mutant. Like P475D, P475E demonstrates strong inward currents at negative voltages (FIGS. 4A and 4B are similar to FIGS. 3A and 3B). The inward currents cannot be attributed to background and leak currents. Background and leak currents have been subtracted from the Shaker K⁺ channel current by blocking Shaker with saturating concentrations (1 μM) of agitoxin2 and then subtracting the unblocked currents (leak and background currents) from the total currents measured in the absence of agitoxin2. Thus, P475D is a constitutively open voltage-gated K⁺ channel. Note however that P475D and P475E are distinguishable in terms of ionic conductance and other channel characteristics.

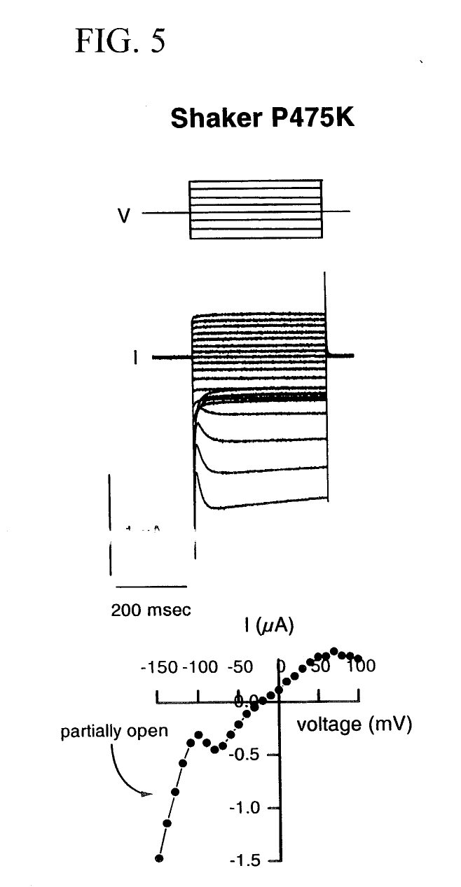

FIG. 5 presents voltage-current data for the Shaker P475K mutant. Like P475D and P475E, P475K demonstrates inward currents at negative voltages (FIGS. 5A and 5B are similar to FIGS. 3A and 3B, and FIGS. 4A and 4B). The inward currents are smaller in magnitude than those observed with P475D and P475E, but nevertheless are present and persist in spite of strong hyperpolarizing voltages (up to −150 millivolts). The inward currents cannot be attributed to background and leak currents, because these have been subtracted by blocking Shaker with saturating concentrations (1 μM) of agitoxin2 and then subtracting the unblocked currents (leak and background currents) from the total currents measured in the absence of agitoxin2. Thus, P475K is a constitutively open voltage-gated K⁺ channel. Again, its characteristics are distinguishable not only from wild type, but also from other P475 mutants.

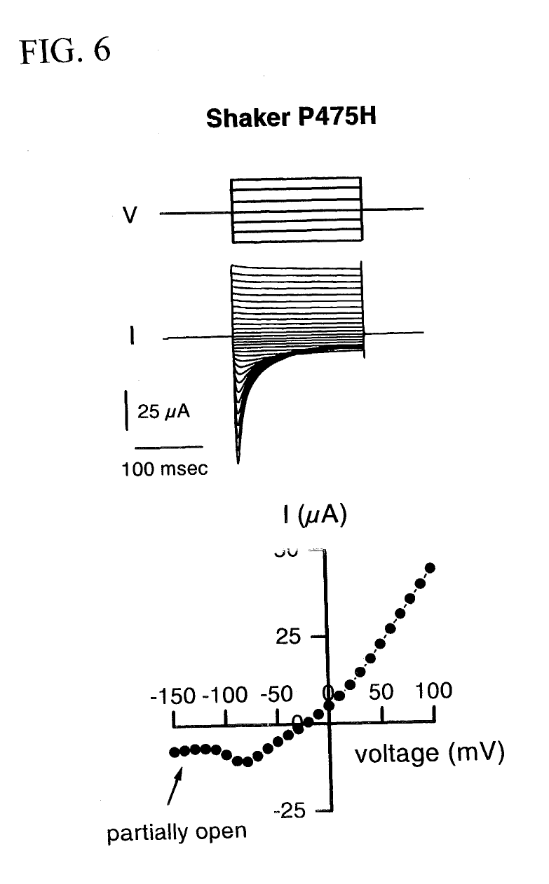

FIG. 6 presents voltage-current data for the Shaker P475H mutant. Like P475D, P475E, and P475K, P475H demonstrates inward currents at negative voltages (FIGS. 6A and 6B). The inward currents are of somewhat smaller magnitude than those observed with P475D and P475E, but nevertheless are present and persist in spite of strong hyperpolarizing voltages (up to −150 millivolts). In FIG. 6B, the shape of the curve at voltages more negative than −60 millivolts shows that the channel begins to close at hyperpolarizing voltages, but does not do so completely. The inward currents cannot be attributed to background and leak currents, because these have been subtracted by blocking Shaker with saturating concentrations (1 μM) of agitoxin2 and then subtracting the unblocked currents (leak and background currents) from the total currents measured in the absence of agitoxin2. Thus, P475H is a constitutively open voltage-gated K⁺ channel. Its characteristics are distinguishable not only from wild type, but also from other P475 mutants.

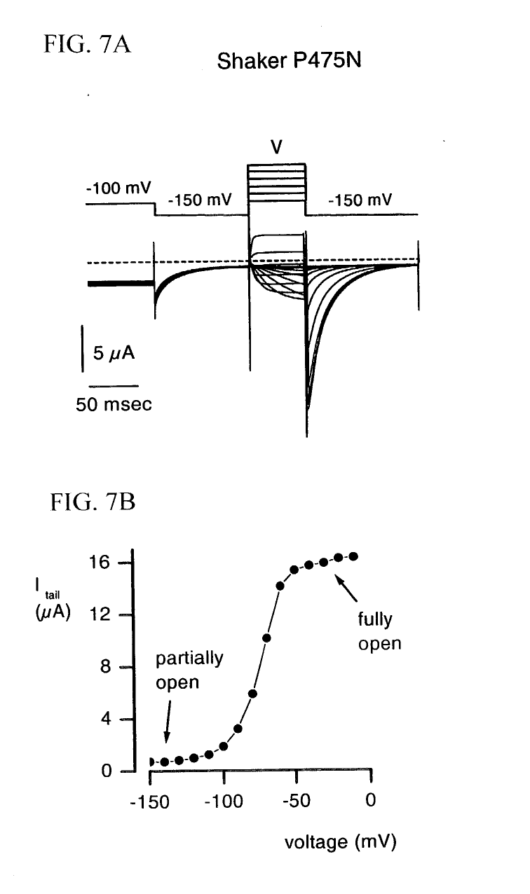

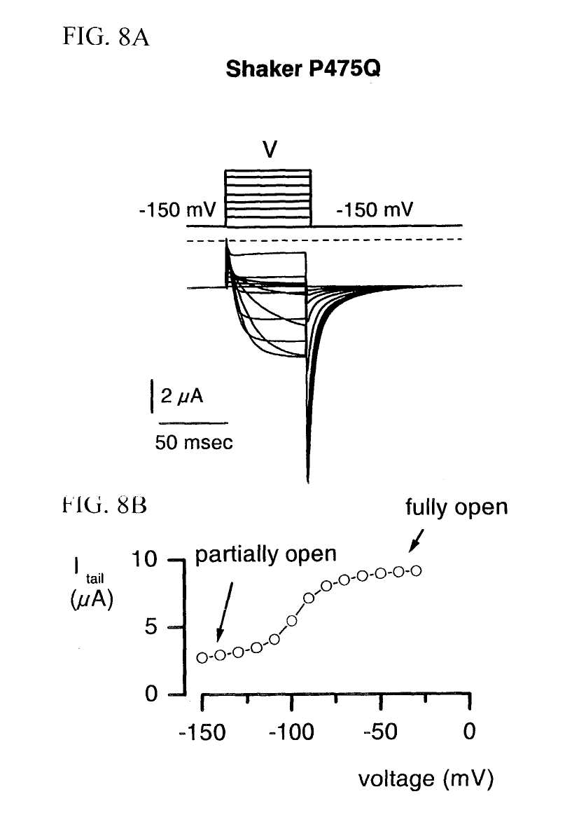

FIG. 7A presents voltage-current data for the Shaker P475N mutant. Like P475D, P475E, P475K and P475H, P475N demonstrates inward currents at negative voltages (FIG. 8A; dotted line marks the zero current position). A large inward current was observed at −100 mV and a moderate current observed at −150 mV. With depolarization an additional current activates during the depolarization and then deactivates upon returning to −150 mV.

FIG. 7B presents tail currents observed with P475N, recorded 2 msec after return from test depolarizing voltage to −150 mV (see Example 3 for discussion of tail currents). The tail current begins at a non-zero level because channels do not close completely, even at the very negative voltage of −150 mV. With depolarization, the channels are further activated. These data demonstrate that P475N is a constitutively open voltage-gated K⁺ channel. Its characteristics are distinguishable not only from wild type, but also from other P475 mutants.

FIGS. 8A and 8B present voltage-current and tail current data for the Shaker P475Q mutant. Its characteristics are similar to P475N: inward currents at negative voltages (FIG. 8A; dotted line marks the zero current position); and a large inward current observed at −150 mV. With depolarization an additional current activates during the depolarization and then deactivates upon returning to −150 mV. FIG. 8B demonstrates a nonzero tail current even at very negative membrane voltages, and also shows that the channel may be further activated upon depolarization.

In summary, these data establish that several mutations at P475 in the Shaker H4 voltage-gated K⁺ channel alter potassium conductance, producing mutant channels that remain partially open at voltages sufficiently negative to close the wild type Shaker H4 channel. Each channel mutant may be distinguished from the others on the basis of voltage-current relationships and/or tail current characteristics.

### EXAMPLE 5

### Mutation in Mammalian Voltage-Gated K⁺ Channel Produces a Constitutively Open Phenotype

Pro 406 (P406) in Kv2.1 (a Shab family member obtained from rat) is homologous to P475 of the Shaker H4 voltage-gated K⁺ channel (see FIG. 1 and Example 1). To evaluate the effects of mutations at P406, a P406D mutant was constructed as described in Example 2. A cRNA encoding this mutant was injected into Xenopus oocytes, and the electrophysiological characteristics of this channel mutant were determined as described in Examples 3 and 4.

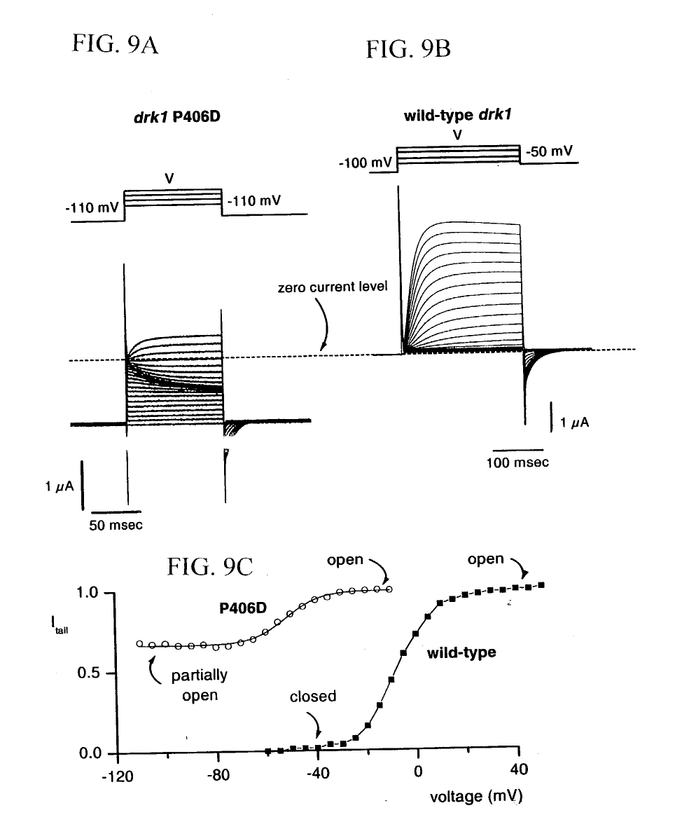

FIG. 9A presents voltage-dependent gating in the wild type Kv2.1 K⁺ channel. Membrane voltage was held at −100 mV and depolarized to different voltages and then returned to −50 mV to elicit a tail current. A standing current at −100 mV was absent because all channels are closed at these negative voltages. Compare the behavior of this mammalian channel to the Shaker H4 K⁺ channel presented in Example 3 and FIG. 3. Neither of these unmutated channels shows a negative (inward) current at negative voltages, indicating that they close completely under these conditions.

FIG. 9B presents voltage-current data from an oocyte expressing a Kv2.1 K⁺ mutant, P406D (in which proline is substituted with aspartate). FIG. 9B, top, shows that membrane voltage was held at −110 mV, depolarized to different voltages, and returned to −110 mV to elicit a tail current. A large inward current observed at −110 mV, and an additional current activates during depolarization and deactivates upon returning to −110 mV (FIG. 9B, bottom; dashed line indicates zero current level). Background and leak currents have been subtracted from the Kv2.1 K⁺ channel current by blocking Kv2.1 with saturating concentrations (1 μM) of agitoxin2 (Garcia et al., 1994) and then subtracting the unblocked currents (leak and background currents) from the total currents measured in the absence of agitoxin2. Thus the standing current observed at −110 mV is not a leak current but a current arising from the mutated Kv2.1 K⁺ channel. Thus, the P406D mutant voltage-gated K⁺ channel does not close completely at negative voltages, and is therefore constitutively open.

FIG. 9C compares tail current activation curves for P406D and wild-type Kv2.1 K⁺ channels. Normalized tail currents are plotted against test voltage for the two Xenopus oocytes shown in 9A and 9B. For P406D the tail current was measured 2 msec after repolarization to −110 mV, and for wild type the tail was measured 2 msec after repolarization to −50 mV.

In P406D the tail current begins at a non-zero level because channels do not close completely, even at the very negative voltage of −110 mV, and channels can be further activated with depolarization. In contrast, the wild-type tail current begins at zero because these channels close completely at −50 mV.

These data establish that a mutation at P406 in the Shab family member Kv2.1 voltage-gated K⁺ channel alters potassium conductance, producing a mutant channel that remains partially open at voltages sufficiently negative to close the wild type Shaker H4 channel. The highly conserved nature of voltage-gated K⁺ channels establishes that similar mutations in other voltage-gated potassium channels will also produce a constitutively open phenotype.

### EXAMPLE 6

### Expression of Heterologous Constitutively Active Voltage-Gated K⁺ Channels Enable SGY1528 to Grow in Low-Potassium Media

The SGY1528 strain of Saccharomyces cerevisiae lacks the two yeast membrane potassium transporters trk1 and trk2. Consequently, the strain grows poorly in low-potassium media (0.5-10 mM). However, expression of a constitutively open voltage-gated K⁺ channel in SGY1528 cells confers ability to grow in low potassium.

SGY1528 cells are transformed with a nucleic acid encoding a constitutively open voltage-gated K⁺ channel operably linked to a promoter that is functional in yeast. The nucleic acid may be a DNA expression plasmid, and may be introduced into SGY1528 cells using any of a number of standard transformation techniques known to those with skill in the art. See, e.g., Tang et al., Mol. Biol. Cell 6: 1231-1240 (1995). The plasmid pYES2 (Invitrogen, La Jolla Calif.) is one example of a suitable plasmid for expressing voltage-gated K⁺ channels in yeast.

After SGY1528 are transformed in this manner, their ability to grow in low-potassium media is then assessed. Any constitutively active voltage-gated K⁺ channel may be evaluated. For example, Shab family mutants with P406 mutations, or Shaker family mutants with P475 mutations, may be evaluated. Growth is tested on plates and in liquid culture as follows.

Growth on Plates

SGY1528 cells containing expression plasmids are grown up overnight in minimal yeast media containing 2% glucose and 100 mM KCl. Cells are then collected, washed once in sterile water, and resuspended at 100,000 cells/ml. Equivalent numbers of cells are spotted onto plates containing 7 mM KCl. As a control, equivalent numbers of cells are also spotted onto plates containing 100 mM KCl. Both sets of plates are incubated at 30° C. for 2-3 days, and growth assessed. Cells expressing a constitutively active voltage-gated K⁺ channel grow well on both 7 mM and 100 mM KCl plates. Untransformed SGY1528 cells, or those transformed with plasmids not expressing K⁺ channels and grow well on 100 mM KCl plates, but have significantly impaired growth on 7 mM KCl plates.

Growth in Liquid Culture

Growth characteristics may also be evaluated in liquid culture. SGY1528 cells containing expression plasmids are grown up overnight in minimal yeast media containing 2% glucose and 100 mM KCl. Cells are collected, washed once in sterile water, and resuspended at 100,000 cells/ml. Equivalent numbers of resuspended cells are used to inoculate 100 ml shaker flasks containing 50 ml of yeast media containing 7 mM KCl. Cells are grown at 30° C. with vigorous aeration. At successive time points from about 6-48 hours post-inoculation, the optical density at 600 nm (O.D. 600) is determined on 1 ml aliquots removed from the flasks. Transformed SGY1528 cells expressing constitutively active voltage-gated K⁺ channel exhibit uniform, time-dependent increases in O.D.600. In contrast, untransformed SGY1528 cells show little or no time-dependent increase in O.D.600.

Two additional types of controls may be included in these experiments. As a positive control, SGY1528 cells are transformed with the high-capacity yeast potassium transporter trk1. Trk1-transformed SGY1528 cells exhibit uniform, time-dependent increases in O.D.600, since expression of the trk1 gene in SGY1528 cells restores their ability to grow in low-potassium media. As an additional negative control, SGY1528 cells are transformed with a control plasmid that does not express a K⁺ channel, for example a yeast expression plasmid which does not contain a cDNA. Control plasmid-transformed SGY1528 cells show little or no time-dependent increase in O.D.600.

Different constitutively active K⁺ channel mutants would have distinguishable growth characteristics. For example, P475N confers a slow-growth phenotype on SGY1528 cells grown in low potassium. In contrast, the P475D mutant markedly enhances potassium conductance and confers a fast-growth phenotype that approaches that of wild type Saccharomyces cerevisiae.

### EXAMPLE 7

### Method for Detecting Substances that Modulate Voltage-Gated K⁺ Channels in SGY1528 Cells

As shown in Example 4, transformation of SGY1528 cells with a constitutively active voltage-gated K⁺ channel enables the transformed SGY1528 cells to grow in low-potassium media. This characteristic forms the basis of a high-throughput system for evaluating substances that may modulate voltage-gated K⁺ channel function.

A High Throughput System for Testing Possible Inhibitors of Voltage-Gated K⁺ Channel Function.

SGY1528 cells are transformed with a nucleic acid encoding a constitutively active voltage-gated K⁺ channel operably linked to a promoter, as described in Example 4. As before, SGY1528 cells containing expression plasmids are grown up overnight, washed and resuspended at about 100,000 cells/ml. Equivalent numbers of cells are spotted onto two or more plates containing 7 mM KCl, and two or more plates containing 100 mM KCl. Some of the low and high-potassium plates containing a substance that may inhibit voltage-gated K⁺ channels, and some do not. The four sets of plates are incubated at 30° C. for 2-3 days, and growth assessed.

As in Example 4, cells expressing a constitutively active voltage-gated K⁺ channel grow well on both 7 mM and 100 mM KCl plates. If the substance added to the plates does not inhibit voltage-gated K⁺ channels, no effect on growth will be observed in either low- or high-potassium. However, if the substance inhibits voltage-gated K⁺ channels, poor low-potassium growth will be observed. In other words, the cells will yield the same results as untransformed SGY1528 cells: poor low-potassium growth, normal high-potassium growth.

Untransformed SGY1528 cells, or those transformed with plasmids not expressing K⁺ channels, grow poorly in low-potassium but grow well in high-potassium, regardless of the presence of a voltage-gated K⁺ channel inhibitor.

Table 1 summarizes these results.

TABLE 1
Growth of transformed and untransformed SGY1528 cells.

| | 100 mM KCl | 100 mM KCl | 7 mM KCl | 7 mM KCl |
|---|---|---|---|---|
| | −inhibitor | +inhibitor | −inhibitor | ^{+inhibitor} |
| SGY1528 | +++ | +++ | − | − |
| | | | − | − |
| SGY1528 + | +++ | +++ | − | − |
| pYES2 | | | | |
| SGY1528 + | +++ | +++ | +++ | − |
| pYES2 − P406D | | | | |

−indicates little or no growth;
+++indicates growth approximately equivalent to wild type Saccharomyces cerevisiae cells.

Putative inhibitors of voltage-gated K⁺ channels could also be evaluated in liquid cultures of SGY1528 cells, as described in Example 4. An effective inhibitor of voltage-gated K⁺ channels would retard growth of SGY1528 cells in low-potassium media. The degree of growth retardation would give a semiquantitative estimate of the inhibitor's potency.

Some constitutively open voltage-gated K⁺ channels may produce an alternative phenotype in high K⁺ media. For example, P475E in Shaker H4 may impair growth in high K⁺ (e.g., 100 mM extracellular KCl) media. The persistently large K⁺ leak at resting membrane potentials would produce detrimentally high intracellular potassium levels. This effect makes the assay even more robust, since it allows screening for voltage-gated K⁺ channel inhibitors for their ability to promote growth in high-potassium media.

The SGY1528-based growth assays in this Example are also used to screen for K⁺ channel activators. SGY1528 cells are transformed with a nucleic acid expressing Shaker H4 P475N, P475Q, or equivalent mutation in other voltage-gated K⁺ channel family members. The slow inward K⁺ current that these mutants produce at resting membrane voltages is insufficient to restore full growth in low K⁺ media. For example, SGY1528+pYES2-P475N has either (−) growth or trace growth in 7 mM KCl (in Table 1, compare to SGY1528+pYES2-P406D in 7 mM KCl − inhibitor, which has +++ growth). However, SGY1528+pYES2-P475N has +++ growth if a K⁺ channel activator is present in the media.

### EXAMPLE 8

### Method for Detecting Substances that Modulate Voltage-Gated K⁺ Channels

A mutated nucleic acid expressing a constitutively active voltage-gated K⁺ channel subunit is used to transfect Xenopus oocytes or other suitable eukaryotic cell, such as CHO or HEK 293 cells. Transfection and expression is performed as described in Example 2. Currents are recorded using standard two-microelectrode voltage clamp techniques about three days after transfection, as described in Example 3, and in Baldwin et al., Neuron 7: 471-483 (1991). The drug candidate is added to the bathing solution of the transfected cells and the effect of the drug candidates upon the induced current is determined. Some drug candidates may further enhance potassium conductance, whereas others may reduce potassium conductance. Other effects of possible therapeutic significance are possible; for example, a drug may affect the voltage-gated K⁺ channel's activation or inactivation properties, or affect its response to voltage changes.

### EXAMPLE 9

### Assay to Screen for Substances that Modulate Voltage-Gated K⁺ Channels in Xenopus Oocytes or Other Suitable Eukaryotic Cells

A complementary approach to that described in Examples 5-8 is to focus the electrophysiologic studies on compounds identified as potential voltage-gated K⁺ channel modulators in Example 4. Xenopus oocytes or any other suitable eukaryotic cells are transformed with a mutated nucleic acid expressing a constitutively active voltage-gated K⁺ channel. Transformation and expression are performed as described in Example 2. Currents are recorded using standard two-microelectrode voltage clamp techniques about three days after transfection, as described in Example 3. Potential voltage-gated K⁺ channel modulators identified in Example 4 are added to the bathing solution of transfected cells, the effect of the drug candidates upon the induced current is determined. As in Example 5, a broad range of voltage-gated K⁺ channel properties are evaluated, and the compound may affect any one or more of these properties. Promising lead compounds are selected for further study in preclinical trials.

### EXAMPLE 10

### Assays to Screen for Substances that Modulate Voltage-Gated K⁺ Channels Using Intracellular Markers of Potassium Concentration

Another option for screening substances that may modulate voltage-gated K⁺ channels relies on intracellular markers of potassium concentration. Examples of such markers are described in U.S. Pat. No. 5,948,906, which is incorporated by reference. Intracellular signals from these markers are measured in the presence of candidate compounds that are being studied as potential modulators of potassium channel conductance.

SGY1528 cells and SGY1528 cells transformed with a constitutively active voltage-gated K⁺ channel, are loaded with a fluorescent potassium indicator dye such as PBFP (florescent potassium-binding benzofuran pthalate; U.S. Pat. No. 5,948,906). Any constitutively active voltage-gated K⁺ channel is suitable, and yields results that are distinguishable from other constitutively active voltage-gated K⁺ channels. Fluorescence excitation and emission wavelengths are optimized by standard techniques known to those with skill in the art, and are expected to be about 345-350 nm and 500-550 nm, respectively.

In low-potassium media, SGY1528 cells show weak fluorescence, whereas SGY1528 cells expressing P406D or P475D show stronger fluorescence. The fluorescence intensity ratio (e.g., the intracellular intensity of fluorescence observed with P406D-transformed cells divided by intracellular fluorescence intensity of untransformed cells) is determined using fluorescence spectroscopy. A substance that inhibits voltage-gated K⁺ channels decreases this ratio (by decreasing intracellular fluorescence), whereas an activator increases the ratio. Alternatively, relative fluorescence intensity may be evaluated with fluorescence microscopy, confocal microscopy, or any other suitable fluorescence-based technique, either quantitatively or qualitatively.

Intracellular potassium markers are useful with a broad range of other eukaryotic cells. For example, Xenopus oocytes, CHO cells, or HEK 293 cells may be substituted for SGY1528 cells.

Another option for screening substances for voltage-gated K⁺ channel modulation relies on intracellular markers of voltage. Examples of such fluorescent voltage markers are described in U.S. Pat. No. 5,661,035, which is incorporated by reference, and Tsein et al., Biophysical Journal 69: 1272-1280, 1995, also incorporated by reference.

SGY1528 cells and SGY1528 cells transformed with a constitutively active voltage-gated K+ channel, are loaded with a fluorescent voltage marker such as FLOX4 or FLOX6 as described in incorporated U.S. Pat. No. 5,661,035 and Tsein et al., Biophysical Journal 69: 1272-1280, 1995. Any constitutively active voltage-gated K+ channel is suitable, and yields results that are distinguishable from other constitutively active voltage-gated K+ channels. Fluorescence excitation and emission wavelengths are optimized by standard techniques known to those with skill in the art, and are about 450-490 nm and 505-560 nm, respectively.

In low-potassium media, SGY1528 cells show weak fluorescence, whereas SGY1528 cells expressing P406D or P475D show stronger fluorescence. The fluorescence intensity ratio (e.g., the intensity of cellular fluorescence observed with P406D-transformed cells divided by cellular fluorescence intensity of untransformed cells) is determined. A putative voltage-gated K⁺ channel inhibitor decreases this ratio, whereas an activator increases the ratio. Alternatively, relative fluorescence intensity may be evaluated with fluorescence microscopy, confocal microscopy, or any other suitable fluorescence-based technique.

Fluorescent voltage markers are useful with a broad range of other eukaryotic cells. For example, Xenopus oocytes, CHO cells, or HEK 293 cells may be substituted for SGY1528 cells.

### EXAMPLE 11

### Transgenic Animals

Mutant organisms that express constitutively active voltage-gated K⁺ channels are useful for research. Such mutants allow insight into the physiological and/or pathological role of voltage-gated K⁺ channels of healthy and/or pathological organisms. These mutants are “genetically engineered,” meaning that information in the form of nucleotides has been transferred into the mutant's genome at a location, or in a combination, in which it would not normally exist.

Mutants may be, for example, produced from mammals, such as mice, that express a constitutively open voltage-gated K⁺ channel derived from a Shaker, Shab, Shal, or Shaw family member. Such mutants are made by introducing a nucleic acid expressing a constitutively open voltage-gated K⁺ channel into the organism under the control of a constitutive or inducible or viral promoter, such as the mouse mammary tumor virus (MMTV) promoter or the whey acidic protein (WAP) promoter or the metallothionein promoter. The promoter may be relatively tissue specific, for example the α-myosin heavy chain promoter for muscle expression, or the insulin promoter for pancreatic β-cell expression. See U.S. Pat. No. 5,298,422 and U.S. Pat. No. 5,824,840, which are incorporated by reference. Virtually any tissue can be targeted by the use of such relatively tissue-specific promoters.

A mutant mouse expressing a constitutively active voltage-gated K⁺ channel may be made by constructing a plasmid having the desired a constitutively active voltage-gated K⁺ channel gene driven by a promoter, such as the mouse mammary tumor virus (MMTV) promoter, a myosin heavy chain promoter, or insulin promoter. This plasmid may be introduced into mouse oocytes by microinjection. The oocytes are implanted into pseudopregnant females, and the litters are assayed for insertion of the transgene. Multiple strains containing the transgene are then available for study.

An inducible system may be created in which the subject expression construct is driven by a promoter regulated by an agent that can be fed to the mouse, such as tetracycline. Such techniques are well known in the art.

Transgenic organisms expressing constitutively open voltage-gated K⁺ channels are then used to evaluate the physiologic and pathologic impact of voltage-gated K⁺ channel dysfunction, as well as the potential impact of substances that inhibit or activate voltage-gated K⁺ channels. For example, a transgenic mouse expressing a Kv2.1 P406D mutant channel under control of the α-myosin heavy chain promoter may be prone to abnormal heart rhythms. Such a genetically engineered mutant organism would be a powerful tool for studying the origins and possible treatments of abnormal heart rhythms.

### EXAMPLE 12

### Other Mutations that Produce a Constitutively Open Phenotype and Their Utility

A broad range of mutations in both the pore domain and the voltage sensing domain produce a constitutively open phenotype in Drosophila and mammalian voltage-gated K⁺ channels. Mutations are made and expressed according to methods presented in Example 2. Mutations are evaluated for a constitutively open phenotype using the electrophysiological methods of Examples 3-5, and/or by screening for low-potassium growth in transformed SGY1528 cells as described in Example 6.

For example, mutations in the S6 transmembrane domain at sites other than the PVP motif produce a constitutively open phenotype. By way of example, in Shaker H4 amino acid residues at position 470, 474, and 478 contribute to the inner lining of the potassium ion pore. Mutations that convert these or other pore residues to hydrophilic or charged residues produce a constitutively open phenotype. Similar results are obtained at homologous residues of Shab, Shaw, and Shal subfamily members. Mutations that increase hydrophilicity or charge in hydrophobic residues of S6 also produce a constitutively open phenotype. For example, mutation of any Shaker H4 residue between Ala 463-Phe 480 (or the homologous residue in Shaker, Shab, Shal, and Shaw subfamily members) to Asp, Glu, Lys, His, Asn, or Gln produces a constitutively open phenotype.

Mutations in or near the pore helix produce a constitutively open phenotype. This region is approximately bounded by Phe 426 and methionine 440 of Shaker H4, or the homologous residues in mammalian Shaker, Shab, Shaw, and Shal subfamily members. For example, in rat Shab subfamily member Kv2.1, the pore helix is approximately bounded by Phe 357 and Met 371. Particular pore helix mutations that produce a constitutively open phenotype in Shaker include those that substitute hydrophilic or charged residues for: Phe 426, Ser 428, Ile 429, Pro 430, Ala 432, Phe 433, Trp 434, Trp 435, Ala 436, Val 437, Val 438, Thr 439, and/or Met 440. Hydrophilic or charged mutations at homologous residues in Shaker, Shab, Shal, and Shaw subfamily also produce a constitutively open phenotype.

Mutations in or near the S5 transmembrane domain of voltage-gated K⁺ channels produce a constitutively open phenotype. In Shaker, the S5 transmembrane domain extends approximately from Ser 392 through Ala 419. Corresponding residues Shaker, Shah, Shaw, and Shal subfamily are readily identified by those skilled in the art through amino acid sequence homology. The S5 transmembrane domain is highly hydrophobic, and virtually any S5 mutation that increases charged or hydrophilicity will produce a constitutively open phenotype. For example, any residue from Leu 398 through Ala 417 may be mutated to either Asp or Glu to produce the constitutively open phenotype. Moreover, at each position, a broad range of other substitutions also produce the constitutively open phenotype.

Mutations in or near S1-S4 voltage sensing domain of voltage-gated K+ channels produce a constitutively open phenotype. For example, the S4 transmembrane domain contains a series of positively charged amino acid residues that play a key role in voltage sensing; these are mutated to uncharged or negatively charged residues to alter voltage sensing and produce a constitutively open phenotype.

Any constitutively open voltage-gated K⁺ channel may be used in Examples 4-11. Such mutations may be either in the pore domain or the voltage sensing domain, and the precise mutation or mutations that produce the constitutively open phenotype in general is not of critical importance to the practice of the invention as described in these examples.

The degeneracy of the genetic code further widens the scope of the present invention as it enables major variations in the nucleotide sequence of a DNA molecule while maintaining the amino acid sequence of the encoded protein. For example, amino acid residue 463 in Shaker H4 is Ala. This is encoded in the Shaker H4 cDNA by the nucleotide codon triplet GCC. Because of the degeneracy of the genetic code, three other nucleotide codon triplets, GCG, GCT and GCA, also code for alanine. Thus, the nucleotide sequence of the Shaker H4 cDNA could be changed at this position to any of these three codons without affecting the amino acid composition of the encoded protein or the characteristics of the protein. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein also comprehended by this invention.

One skilled in the art will recognize that the DNA mutagenesis techniques described above may be used not only to produce variant DNA molecules, but will also facilitate the production of proteins which differ in certain structural aspects from voltage-gated K⁺ channel proteins such as those of the Shaker, Shab, Shal, and Shaw subfamilies, yet which proteins are clearly derivative of these proteins and which maintain most or all of the essential functional characteristics of the protein as defined above. Newly derived proteins may also be selected in order to obtain variations on the characteristic of the protein, as will be more fully described below. Such derivatives include those with variations in amino acid sequence including minor deletions, additions and substitutions.

While the site for introducing an amino acid sequence variation is predetermined, the mutation per se need not be predetermined. For example, in order to optimize the performance of a mutation at a given site, random mutagenesis may be conducted at the target codon or region and the expressed protein variants screened for optimal activity. Techniques for making substitution mutations at predetermined sites in DNA having a known sequence as described above are well known.

Amino acid substitutions are typically of single residues; insertions usually will be on the order of about from 1 to 10 amino acid residues; and deletions will range about from 1 to 30 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure.

Substitutional variants are those in which at least one residue in the amino acid sequence has been removed and a different residue inserted in its place. Such substitutions generally are conservative when it is desired to finely modulate the characteristics of the protein. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gin; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Substantial changes in function or immunological identity are made by selecting substitutions that are less conservative than those listed above, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives of the mutated voltage-gated K⁺ channel proteins by transient expression of the protein in question in cells such as Xenopus oocytes or SGY1528 cells as described in Examples 3-10.

### EXAMPLE 13

### Constitutively Open Voltage-Gated K+ Channels Derived from eag, elk, and erg Subfamilies

The principles of the present invention also apply to the eag, elk, and erg subfamilies of voltage-gated K+ channels. Mutations at sites that produce a constitutively open voltage-gated K+ channel in a Shaker, Shab, Shal, or Shaw subfamily members are identified. For example, several mutations of P475 of Shaker H4 and P406 of the Shab subfamily had been identified in the present invention as producing a constitutively open phenotype. The corresponding residue or residues of an eag, elk, or erg subfamily member (referred to here as the “target residues”) are identified by sequence alignment/homology programs, such as BLAST 2.0 (available at the NCBI website) or MacVector 6.5.3 (Oxford Molecular, Oxford UK). Approximately 10-15 residues on either side of the target residues are mutated individually or in combination to charged or hydrophilic residues, using the methods of Example 2. For example, the region that approximately corresponds to the PVP motif's location is: in Herg, amino acids 645 to 665 (Trudeau et al., Science 269:92-95, 1995); Meag, 485 to 505 (Warmke et al., Proc. Natl. Acad. Sci. U.S.A. 91, 3438-3442, 1994); Melk2, 490 to 510 (Trudeau et al., Journal of Neuroscience. 19(8):2906-18, 1999) Elk, 490 to 510 (Shi et al., Journal of Physiology. 511: 675-82, 1998).

The electrophysiological effects of the mutations are determined using the methods of Examples 3-5. In addition or alternatively, the ability of a particular eag, elk, or erg subfamily mutant to rescue low potassium growth of SGY1528 cells is determined, using methods similar to those described in Example 6. Once eag, elk, or erg subfamily members with constitutively open phenotypes are identified, substances that may modulate these channels are tested using methods similar to those described in Examples 7-10.

Homologs of the disclosed human channel proteins typically possess at least 60% sequence identity counted over full-length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). This approach enables the comparison of motifs such as the S5 and S6 transmembrane domains, and the S5/S6 linker, between subfamilies of voltage-gated K+ channels. When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, at least 75%, at least 80%, at least 90%, at least 95%, at least 98%, or at least 99% sequence identity. When less than the entire sequence is being compared for sequence identity, homologs will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI website.

In view of the many possible embodiments to which the principles of the invention may be applied, it should be recognized that the illustrated embodiments are examples of the invention, and should not be taken as a limitation on the scope of the invention. Rather, the scope of the invention is defined by the following claims. We therefore claim as our invention all that comes within the scope and spirit of these claims.

We claim:

1. A purified protein having voltage-gated K⁺ channel activity, the protein mutated so that it produces a constituitively open voltage-gated K⁺ channel.

2. The mutated purified protein of claim 1, wherein the mutated purified protein is selected from the group consisting of: Shaker family, Shab family, Shal family, and Shaw family.

3. The mutated purified protein of claim 1, wherein prior to mutation the protein contained a PVP motif in its pore domain.

4. The mutated purified protein of claim 3, wherein the mutation comprises an amino acid substitution at the second Pro of the PVP motif.

5. The mutated purified protein of claim 4, wherein the mutation comprises an amino acid substitution selected from the group consisting of: Asp, Glu, Lys, His, Asn, and Gln.

6. The mutated purified protein of claim 3,wherein the mutation comprises an amino acid substitution selected from the group consisting of: Arg, Tyr, Phe, Trp, Tyr, Met, Gly, Val, Ile, Cys, Ser, and Ala.

7. The mutated purified protein of claim 2, wherein the mutated purified protein is derived from the Shaker family, and the mutations comprises those that produce an amino acid substitution at Pro 475.

8. The mutated purified protein of claim 7, wherein the mutation comprises an amino acid residue selected from the group consisting of: Asp, Glu, Lys, His, Asn, and Gln.

9. The mutated purified protein of claim 7,wherein the mutation comprises an amino acid substitution selected from the group consisting of: Arg, Tyr, Phe, Trp, Tyr, Met, Gly, Val, Ile, Cys, Ser, and Ala.

10. The mutated purified protein of claim 2, wherein the mutated purified protein is derived from the Shab family, and the mutation comprises those that produce an amino acid substitution at Pro 406.

11. The mutated purified protein of claim 10, wherein the mutation comprises an amino acid substitution selected from the group consisting of: Asp, Glu, Lys, His, Asn, and Gln.

12. The mutated purified protein of claim 11, wherein the mutation comprises an amino acid substitution selected from the group consisting of: Arg, Tyr, Phe, Trp, Tyr, Met, Gly, Val, Ile, Cys, Ser, and Ala.

13. The mutated purified protein of claim 2, wherein the nucleic acid is derived from the Shal family, and the mutation comprises those that produce an amino acid substitution at Pro 404.

14. The mutated purified protein of claim 13, wherein the mutation comprises an amino acid substitution selected from the group consisting of Asp, Glu, Lys, His, Asn, and Gln.

15. The mutated purified protein of claim 14,wherein the mutation comprises an amino acid substitution selected from the group consisting of: Arg, Tyr, Phe, Trp, Tyr, Met, Gly, Val, Ile, Cys, Ser, and Ala.

16. The mutated purified protein of claim 2, wherein the nucleic acid is derived from the Shaw family, and the mutation comprises those that produce an amino acid substitution at Pro 410.

17. The mutated purified protein of claim 16, wherein the mutation comprises an amino acid substitution selected from the group consisting of: Asp, Glu, Lys, His, Asn, and Gln.

18. The mutated purified protein of claim 16,wherein the mutation comprises an amino acid substitution selected from the group consisting of: Arg, Tyr, Phe, Trp, Tyr, Met, Gly, Val, Ile, Cys, Ser, and Ala.

19. A cell expressing the mutated purified protein of claim 1.

20. The cell of claim 19, wherein the cell comprises a Xenopus oocyte.

21. The cell of claim 20, wherein expression of the expression of the mutated purified protein enhances growth in a reduced-potassium media.

22. The cell of claim 21, wherein the cell comprises a yeast cell.

23. The cell of claim 22, wherein the yeast cell comprises Saccharomyces cerevisiae.

24. The cell of claim 23, wherein the cell has impaired function of trk1-trk2 potassium transporters.

25. The cell of claim 24, wherein the mutated purified protein is selected from the group consisting of: Shaker family, Shab family, Shal family, and Shaw family.

26. A method for testing a substance to determine whether it modulates voltage-gated K⁺ channel function, comprising:
providing a cell expressing the mutated purified protein of claim 1;
treating the cell with the substance;
detecting any phenotypic or genotypic characteristic of the cell.

27. The method of claim 26, wherein the characteristic comprises voltage-gated K⁺ channel function.

28. The method of claim 27, wherein the voltage-gated K⁺ channel function comprises potassium conductance.

29. The method of claim 27, wherein the voltage-gated K⁺ channel function is selected from the group consisting of: steady-state current and tail current.

30. The method of claim 27, wherein the voltage-gated K⁺ channel function comprises voltage-gating.

31. The method of claim 27, wherein the voltage-gated K⁺ channel function comprises channel activation.

32. The method of claim 27, wherein the voltage-gated K⁺ channel function comprises channel inactivation.

33. The method of claim 26, wherein the characteristic comprises cell growth.

34. The method of claim 33, wherein the cell comprises a yeast cell.

35. The method of claim 34, wherein the yeast cell comprises a Saccharomyces cerevisiae cell having impaired or absent function of the trk1-trk2 potassium transporters.

36. A compound, wherein the compound is identified as having ability to modulate voltage-gated K+ channel function using the method of claim 26.

37. A recombinant nucleic acid molecule comprising a promoter sequence operably linked to a nucleic acid sequence encoding a voltage-gated K⁺ channel polypeptide subunit, the nucleic acid mutated so that it produces a constituitively open voltage-gated K⁺ channel upon expression in a cell.

38. The recombinant nucleic acid molecule of claim 37, wherein the voltage-gated K⁺ channel polypeptide subunit is selected from the group consisting of:
Shaker family, Shab family, Shal family, Shaw family.

39. The recombinant nucleic acid molecule of claim 37, wherein prior to modification the nucleic acid encoded a PVP motif.

40. The recombinant nucleic acid molecule of claim 39, wherein the mutation encodes an amino acid substitution at the second Pro.

41. The recombinant nucleic acid molecule of claim 39, wherein the mutation encodes an amino acid selected from the group consisting of: Asp, Glu, Lys, His, Asn, and Gln.

42. The recombinant nucleic acid molecule of claim 41, wherein the mutation encodes an amino acid selected from the group consisting of: Arg, Tyr, Phe, Trp, Tyr, Met, Gly, Val, Ile, Cys, Ser, and Ala.

43. A cell transformed with the mutated nucleic acid of claim 36.

44. A transgenic animal comprising a recombinant nucleic acid molecule according to claim 36.


SEQUENCE LISTING (9 sequences)

SEQ ID NO: 1 - protein, 90 aa, Streptomyces lividans
HWRAAGAATVLLVIVLLAGSYLAVLAERGAPGAQLITYPRALWWSVETATTVGYGDLYPVTLWGRLVAVVVMVAGITSFGLVTAALATWF

SEQ ID NO: 2 - protein, 90 aa, Drosphila
SMRELGLLIFFLFIGVVLFSSAVYFAEAGSENSFFKSIPDAFWWAVVTMTTVGYGDMTPVGVWGKIVGSLCAIAGVLTIALPVPVIVSNF

SEQ ID NO: 3 - protein, 90 aa, Rattus rattus
SMRELGLLIFFLFIGVVLFSSAVYFAEAEEAESHFSSIPDAFWWAVVSMTTVGYGDMTPVTIGGKIVGSLCAIAGVLTIALPVPVIVSNF

SEQ ID NO: 4 - protein, 90 aa, Drosphila
SYKELGLLMLFLAMGVLIFSSLAYFAEKDEKDTKFVSIPEAFWWAGITMTTVGYGDICPTTALGKVIGTVCCICGVLVVALPIPIIVNNF

SEQ ID NO: 5 - protein, 90 aa, Rattus rattus
SYNELGLLILFLAMGIMIFSSLVFFAEKDEDDTKFKSIPASFWWATITMTTVGYGDIYPKTLLGKIVGGLCCIAGVLVIALPIPIIVNNF

SEQ ID NO: 6 - protein, 89 aa, Drosphila
SAKELTLLVFFLVLGIVIFASLVYYAERNPHNDFNSIPLGLWWALVTMTTVGYGDMAPKTYIGMFVGALCALAGVLTIALPVPVIVSNF

SEQ ID NO: 7 - protein, 89 aa, Rattus rattus
STNEFLLLIIFLALGVLIFATMIYYAERSEHTHFKNIPIGFWWAVVTMTTLGYGDMYPQTWSGMLVGALCALAGVLTIAMPVPVIVNNF

SEQ ID NO: 8 - protein, 90 aa, Drosphila
CASELGFLVFSLAMAIIIFATVMFYAEKNVNGTNFTSIPAAFWYTIVTMTTLGYGDMVPETIAGKIVGGVCSLSGVLVIALPVPVIVSNF

SEQ ID NO: 9 - protein, 90 aa, Rattus rattus
CASELGFLLFSLTMAIIIFATVMFYAEKGSSASKFTSIPAAFWYTIVTMTTLGYGDMVPKTIAGKIFGSICSLSGVLVIALPVPVIVSNF